(19) United States
(12) Patent Application Publication
(10) Pub. No.: US 2026/0306504 A1
(43) Pub. Date: Oct. 8, 2026

(54) METHODS AND PHARMACEUTICAL COMPOSITIONS COMPRISING D1-LIKE DOPAMINE RECEPTOR AGONIST FOR THE TREATMENT OF AUTOSOMAL DOMINANT POLYCYSTIC KIDNEY DISEASE

(71) Applicants: INSTITUT NATIONAL DE LA SANTÉ ET DE LA RECHERCHE MÉDICALE, PARIS (FR); CENTRE HOSPITALIER UNIVERSITAIRE DE ROUEN, ROUEN (FR); UNIVERSITÉ DE ROUEN NORMANDIE, Mont-Saint-Aignan (FR)

(72) Inventors: Jérémy BELLIEN, ROUEN (FR); Dominique GUERROT, ROUEN (FR)

(21) Appl. No.: 19/108,308

(22) PCT Filed: Sep. 5, 2023
(86) PCT No.: PCT/EP2023/074334

§ 371 Date: Mar. 3, 2025

(30) Foreign Application Priority Data
Sep. 6, 2022 (EP) 22306320.7

(51) Int. Cl. A61K 31/55; A61K 31/381; A61P 13/12

(52) U.S. Cl. CPC A61K 31/55; A61K 31/381; A61P 13/12

(57) ABSTRACT

Autosomal dominant polycystic kidney disease (ADPKD) is a hereditary disease affecting 1 in 1,000 to 1 in 2,500 individuals worldwide. The inventor's study performed in 6 patients with ADPKD supports the hypothesis that the stimulation of the dopaminergic system, in particular D1-like family of dopamine receptors, may improve endothelial function by restoring cilia length and mechanotransductory capacity. The inventors carefully assess whether the deficiency in endothelial polycystin-1 and cilia promotes or potentiates both cardiovascular and renal alterations, to explore the mechanisms involved, and to propose and test new pharmacological approaches, in particular targeting the dopaminergic system, to prevent complications of ADPKD. The present invention relates to a method of treating autosomal dominant polycystic kidney disease (ADPKD) in a subject in need thereof comprising a step of administering said subject with a therapeutically effective amount of a D1-like family of dopamine receptors agonist through a continuous systemic delivery.

### FIELD OF THE INVENTION

The present invention is in the field of kidney disease. The present invention relates to methods and pharmaceutical compositions for the treatment of autosomal dominant polycystic kidney disease using a D1-like family of dopamine receptors agonist through a continuous systemic delivery.

### BACKGROUND OF THE INVENTION

Autosomal dominant polycystic kidney disease (ADPKD) is a hereditary disease affecting 1 in 1,000 to 1 in 2,500 individuals worldwide. ADPKD patients present a progressive impairment of kidney function related to the development of multiple renal cysts, typically leading to end-stage renal disease around the age of 55 to 65 years. Irrespective of decreased kidney function, ADPKD is also characterized by an increase in cardiovascular diseases. In particular, ADPKD patients develop arterial hypertension at an early stage of the disease, as well as various left ventricular hypertrophy, cardiac valvulopathies, intracranial aneurysms, and aortic dissections^{(1,2)}. In addition, patients with ADPKD display an altered cardiovascular adaptation to exercise⁽³⁾ and also after surgical creation of arterio-venous fistula (AVF) as a native vascular access for hemodialysis in end-stage renal disease⁽⁴⁾.

Genetic mutations in polycystin 1 (PKD1) are responsible for the majority of resolved cases of ADPKD (approximately 85%, >2 000 pathogenic mutations described in the ADPKD mutation database [http://pkdb.mayo.edu]), with Polycystin-2 (PKD2) (10%) and to a lesser extent GANAB and DNAJB11 mutations. PKD1 and PKD2 respectively encode polycystin-1 and polycystin-2. Polycystin-1 is a transmembrane protein with a large extracellular domain acting as a sensor of mechanical stimuli, with an intracellular domain that interacts with the transient receptor potential channel polycystin-2 (TRPP2), assembled at the membrane in a 1:3 ratio⁽⁵⁾. These proteins form a complex at the primary cilium of renal epithelial cells, sensing flow variation and promoting calcium entry to control cell function and proliferation. The alteration in this pathway contributes to promote cystogenesis in ADPKD patients⁽⁶⁾. Moreover, increasing evidence suggests that the cardiovascular complications of ADPKD are a direct consequence of the presence of abnormal polycystins in vascular cells^{(7, 8, 9)}. In particular, calcium-dependent endothelial NO-synthase activation in response to fluid shear stress is abolished in polycystin-1 or polycystin-2 deficient endothelial cells. The inventors confirmed this finding in vivo in humans by demonstrating that the endothelium-dependent flow-mediated dilatation during sustained flow conditions is profoundly reduced in ADPKD patients, due to the complete loss of NO synthesis (0). Restoring the function of the vascular endothelium may thus represent a unique opportunity to prevent the complications of ADPKD.

In this context, the inventors assess the impact of chronic administration of rotigotine on endothelial function, peripheral and central hemodynamics in ADPKD patients and to obtain specific mechanistic insights by analyzing endothelial Pkd1-deficient mice.

### SUMMARY OF THE INVENTION

The present invention relates to the use of a continuous systemic delivery of D1-like family of dopamine receptors agonist for the treatment of autosomal dominant polycystic kidney disease (ADPKD). In particular, the present invention is defined by the claims.

### DETAILED DESCRIPTION OF THE INVENTION

Altered polycystin-mediated endothelial flow mechanosensitivity contributes to the development of hypertension and cardiovascular complications in patients with autosomal dominant polycystic kidney disease (ADPKD). Stimulation of endothelial type 5 dopamine receptors (DR5) can acutely compensate for the endothelial consequences of polycystin deficiency, but the chronic impact of this approach has to be evaluated in ADPKD.

The inventor's study performed in patients with ADPKD supports the hypothesis that the stimulation of the dopaminergic system, in particular D1-like family of dopamine receptors, may improve endothelial function by restoring cilia length and mechanotransductory capacity⁽¹⁰⁾. In this context, the inventors carefully assess whether the deficiency in endothelial polycystin-1 and cilia promotes or potentiates both cardiovascular and renal alterations, to explore the mechanisms involved, and to propose and test new pharmacological approaches, in particular targeting the dopaminergic system, to prevent complications of ADPKD. Indeed, the inventors demonstrate that in ADPKD patients chronic administration of rotigotine improves conduit artery endothelial function through the restoration of flow-induced NO release as well as hemodynamics, and confirms that endothelial DR5 activation represents a promising pharmacological approach to prevent cardiovascular complications of ADPKD.

Accordingly, the first object of the present invention relates to a method of treating autosomal dominant polycystic kidney disease (ADPKD) in a subject in need thereof comprising a step of administering said subject with a therapeutically effective amount of a D1-like family of dopamine receptors agonist through a continuous systemic delivery.

As used herein, the term “subject” denotes a mammal, such as a rodent, a feline, a canine, and a primate. Particularly, the subject according to the invention is a human. More particularly, the subject according to the invention has or is susceptible to have kidney disease, in particular ADPKD. As used herein, the term “subject” encompasses “patient” As used herein, the term “autosomal dominant polycystic kidney disease” (ADPKD) has its general meaning in the art and refers to an inherited condition that causes small fluid-filled sacs called cysts to develop in the kidneys. ADPKD is caused by a genetic fault that disrupts the normal development of some of the cells in the kidneys and causes cysts to grow. The affected genes in ADPKD are: polycystin 1 (PKD1), which accounts for 85% of cases polycystin 2 (PKD2) which accounts for 15% of cases. PKD1, which is the protein encoded by the PKD1 gene, is present on the cilia and is thought to sense the flow with its large extracellular domains, activating the calcium channels associated with PKD2, the product of gene PKD2, as a result of the genetic setting of ADPKD as explained in the genetics sub-section above. The different symptoms and signs are, but are not limited to acute loin pain, blood in the urine, ballotable kidneys, subarachnoid hemorrhage (berry aneurysm), hypertension, associated liver cysts, uremia due to kidney failure, anemia due to chronic kidney disease, increase RBC or erythropoietin secretion.

As used herein, “continuous systemic delivery” means the administration continuous of a drug product systemically in the body of the patient. Continuous systemic delivery is preferred in order to achieve prolonged and relatively uniform blood concentrations of the drug.

As used herein, “flux” (also called “permeation rate”) is defined as the absorption of a drug through skin or mucosal tissue, and is described by Fick's first law of diffusion:

J=−D(dCm/dx)
where J is the flux in g/cm2/sec, D is the diffusion coefficient of the drug through the skin or mucosa in cm2/sec and dCm/dx is the concentration gradient of the drug across the skin or mucosa.

In some embodiment, the continuous systemic delivery is administered with a pump.

In some embodiment, the D1-like family of dopamine receptors agonist is typically administered via a pump (e.g. a portable infusion pump) that delivers D1-like family of dopamine receptors agonist boluses at specific intervals. Other suitable pumps are e.g. disclosed in the international patent application published under reference WO2007041386 or in U.S. Pat. Nos. 4,722,734; 5,013,293; 5,312,325; 5,328,454; 5,336,168; and 5,372,579.

As used herein, the term “pump” is intended to include any number of drug delivery systems which are capable of dispensing a fluid to a user upon activation. Such drug delivery systems include, for example, injection systems, infusion pumps, bolus injectors, and the like.

Drug pump is configured such that, upon activation by a user by depression of the activation mechanism, the drug pump is initiated to: insert a fluid pathway into the user; enable, connect, or open necessary connections between a drug container, a fluid pathway, and a sterile fluid conduit; and force drug fluid stored in the drug container through the fluid pathway and fluid conduit for delivery into a user. One or more optional safety mechanisms may be utilized, for example, to prevent premature activation of the drug pump.

The drug pump is capable of delivering a range of drugs with different viscosities and volumes. The drug pump is capable of delivering a drug at a controlled flow rate (speed) and/or of a specified volume. In one embodiment, the drug delivery process is controlled by one or more flow restrictors within the fluid pathway connection and/or the sterile fluid conduit. In other embodiments, other flow rates may be provided by varying the geometry of the fluid flow path or delivery conduit, varying the speed at which a component of the drive mechanism advances into the drug container to dispense the drug therein, or combinations thereof.

In some embodiment, the pump is an osmotic pump.

As used herein, the term “osmotic pump” relate to miniature infusion pumps for the continuous dosing of laboratory animals. These minipumps provide researchers with a method for controlled and continuous agent delivery in vivo. Osmotic pumps can be used for systemic administration when implanted subcutaneously or intraperitoneally. They can be attached to a catheter to provide targeted delivery for intravenous, intraarterial, intracerebral, or cranial/calvarial infusion. The pumps can be used to target delivery to a variety of sites including direct substance administration to cord, spleen, liver, organ or tissue transplants. A single pump may provide up to four weeks of infusion.

As used herein, “needle” is intended to refer to a variety of needles including but not limited to conventional hollow needles, such as a rigid hollow steel needles, and solid core needles more commonly referred to as a “trocars.” In a preferred embodiment, the needle is a gauge solid core trocar and in other embodiments, the needle may be any size needle suitable to insert the cannula for the type of drug and drug administration (e.g., subcutaneous, intramuscular, intradermal, etc.) intended. A sterile boot may be utilized within the needle insertion mechanism. The sterile boot is a collapsible sterile membrane that is in fixed engagement at a proximal end with the manifold and at a distal end with the base. In at least on embodiment, the sterile boot is maintained in fixed engagement at a distal end between base and insertion mechanism housing. Base includes a base opening through which the needle and cannula may pass-through during operation of the insertion mechanism, as will be described further below. Sterility of the cannula and needle are maintained by their initial positioning within the sterile portions of the insertion mechanism. Specifically, as described above, needle and cannula are maintained in the sterile environment of the manifold and sterile boot.

In some embodiment, the continuous systemic delivery is administered with a transdermal drug delivery system.

As used herein, the term “transdermal” refers to delivery, administration or application of a drug by means of direct contact with skin or mucosa. Such delivery, administration or application is also known as dermal, percutaneous, transmucosal and buccal. As used herein, “dermal” includes skin and mucosa, which includes oral, buccal, nasal, rectal and vaginal mucosa.

As used herein, the term “transdermal drug delivery system” refers to a system (e.g., a device) comprising a composition that releases drug upon application to the skin (or any other surface noted above). Thereafter, the drug is dispersed throughout the patient's body by blood circulation. Typically, the transdermal drug delivery system is a substantially non-aqueous, solid form, capable of conforming to the surface with which it comes into contact, and capable of maintaining such contact so as to facilitate topical application without adverse physiological response, and without being appreciably decomposed by aqueous contact during topical application to a subject. Many such systems are known in the art and commercially available, such as transdermal drug delivery patches. In accordance with specific embodiments described herein, the transdermal drug delivery systems comprise a drug-containing polymer matrix that comprises a pressure-sensitive adhesive or bioadhesive and drug, and a face adhesive layer disposed on the skin-contacting side of the drug-containing polymer matrix that is adopted for application to a user's (e.g., a subject's) skin.

In one embodiment, the transdermal drug delivery system is a transdermal patch.

As used herein, “polymer matrix” refers to a polymer composition which contains one or more drugs. In some embodiments, the matrix comprises a pressure-sensitive adhesive polymer or a bioadhesive polymer.

In one embodiment, the transdermal drug delivery system, (e.g. the transdermal patch) consists of a backing layer, a single drug-containing polymer matrix layer, a single face adhesive layer, and, optionally, a release liner that is removed prior to use.

The transdermal drug delivery system (e.g. the transdermal patch) may be packaged or provided in a package, typically a peelable pouch, as is used in the art for transdermal drug delivery systems in general. A wide variety of materials known to those skilled in the art of transdermal drug delivery may be used as pouchstock materials, including Surlyn® packaging resins (ethylene acid copolymers) sold by DuPont®, Wilmington, Del.

### Face Adhesive

The transdermal drug delivery system (e.g. the transdermal patch) described herein may comprise a skin-contacting face adhesive layer, separate from the drug-containing polymer matrix. In some embodiments, the face adhesive comprises a rubber-based polymer, such as a rubber-based pressure sensitive adhesive, such as a polyisobutylene or styrene-isoprene-styrene polymer, optionally formulated with a tackifier or plasticizer to provide pressure-sensitive adhesive properties.

Examples of suitable rubber-based polymers and rubber-based pressure-sensitive adhesives include natural or synthetic polyisoprene, polybutylene, polyisobutylene, styrene-butadiene polymers, styrene-isoprene-styrene block copolymers, hydrocarbon polymers, such as butyl rubber, halogen-containing polymers, such as polyacrylic-nitrile, polytetrafluoroethylene, polyvinylchloride, polyvinylidene chloride, and polychlorodiene, and other copolymers thereof. Further specific examples include polyisobutylene pressure-sensitive adhesive polymers, such as DURO-TAK® 87-6908 (Henkel), which comprises polyisobutylene polymer and tackifier. Other specific examples include styrene-isoprene-styrene block copolymers, such as Kraton® D1111 KT (Kraton Performance Polymers, Inc.).

The face adhesive layer may comprise a single rubber-based pressure-sensitive adhesive, or a blend of two or more adhesives. The face adhesive layer may comprise one or more tackifiers, plasticizing agents (such as mineral oil, hydrogenated hydrocarbon resins, aliphatic resins, rosins, and terpenes), and/or other components selected to confer satisfactory physical stability, chemical stability, and/or skin adhesion, as discussed in more detail below.

The face adhesive layer may comprise at least 90%, at least 95%, at least 96%, at least 97%, at least 98%, or least 99% by dry weight of the one or more rubber-based pressure sensitive adhesives. In this context, “% by dry weight of the one or more rubber-based pressure sensitive adhesives” includes the dry weight of the rubber-based polymer and any tackifiers and/or plasticizers included in the composition.

The face adhesive layer may be applied at a coat weight of from 2 to about 20 mg/cm2, including at from about 5 to about 10 mg/cm2, including 5 mg/cm2, 6 mg/cm2, 7 mg/cm2, 8 mg/cm2, 9 mg/cm2, or 10 mg/cm2, or any coat weight there between.

As used herein, “coat weight” refers to the weight of the layer at issue per unit area of the active surface area of the transdermal drug delivery system (e.g. the transdermal patch).

### Drug-Containing Polymer Matrix

The compositions described herein comprise a polymer matrix that is suitable for use with the therapeutic agent (e.g. the D₁-like family receptor agonist) being formulated. Typical polymers used in a polymer matrix for a transdermal drug delivery system (e.g. the transdermal patch) include pressure-sensitive adhesive acrylic polymers, silicone-containing polymers, and rubber-based polymers (such as polyisobutylene and styrene-isoprene-styrene polymers). Non-limiting examples of suitable polymers are set forth below.

The term “acrylic polymer” is used here as in the art interchangeably with “polyacrylate” “polyacrylic polymer” and “acrylic adhesive”. Suitable acrylic-based polymers include homopolymers, copolymers, terpolymers, and the like of various acrylic acids or esters. In some embodiments, acrylic-based polymers are adhesive polymers. In other embodiments, acrylic-based polymers function as an adhesive by the addition of tackifiers, plasticizers, crosslinking agents or other additives. Often, acrylic polymers used in transdermal drug delivery systems (e.g. the transdermal patch) include polymers of one or more monomers of acrylic acids and other copolymerizable monomers, copolymers of alkyl acrylates and/or methacrylates and/or copolymerizable secondary monomers or monomers with functional groups, and combinations of acrylic-based polymers based on their functional groups. Acrylic-based polymers having functional groups include copolymers and terpolymers which contain, in addition to nonfunctional monomer units, further monomer units having free functional groups, where the monomers can be monofunctional or polyfunctional. By varying the amount of each type of monomer added, the cohesive properties of the resulting acrylic polymer can be changed as is known in the art. In some embodiments, the acrylic polymer is composed of at least 50% by weight of an acrylate or alkyl acrylate monomer, from 0 to 20% of a functional monomer copolymerizable with the acrylate, and from 0 to 40% of other monomers.

Typical acrylate monomers include acrylic acid and methacrylic acid and alkyl acrylic or methacrylic esters such as methyl acrylate, ethyl acrylate, propyl acrylate, amyl acrylate, butyl acrylate, butyl methacrylate, hexyl acrylate, methyl methacrylate, hexyl methacrylate, heptyl acrylate, octyl acrylate, nonyl acrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, glycidyl acrylate, and corresponding methacrylic esters.

Typical non-functional acrylic-based polymers include any acrylic based polymer having no or substantially no free functional groups.

As used herein, “functional monomers or groups” are monomer units typically in acrylic-based polymers which have reactive chemical groups which modify the acrylic-based polymers directly or which provide sites for further reactions. Examples of functional groups include carboxyl, epoxy, hydroxyl, sulfoxyl, and amino groups. Acrylic-based polymers having functional groups contain, in addition to the nonfunctional monomer units described above, further monomer units having free functional groups. As noted above, the monomers can be monofunctional or polyfunctional. Typical carboxyl functional monomers include acrylic acid, methacrylic acid, itaconic acid, maleic acid, and crotonic acid. Typical hydroxyl functional monomers include 2-hydroxyethyl methacrylate, 2-hydroxyethyl acrylate, hydroxymethyl acrylate, hydroxymethyl methacrylate, hydroxyethyl acrylate, hydroxyethyl methacrylate, hydroxypropyl acrylate, hydroxypropyl methacrylate, hydroxybutyl acrylate, hydroxybutyl methacrylate, hydroxyamyl acrylate, hydroxyamyl methacrylate, hydroxyhexyl acrylate, hydroxyhexyl methacrylate. Others include amide-group containing-monomers such as octyl acrylamide, and vinyl group containing monomers, such as vinyl acetate and vinyl pyrrolidone In some embodiments, the functionality of the polymer(s) is selected depending on the drug being formulated, e.g., is selected based on whether the functional group is reactive or non-reactive with a functional group on the drug being formulated.

Suitable acrylic polymers also include pressure-sensitive adhesives which are commercially available, such as the acrylic-based adhesives sold under the trademarks DURO-TAK® and GELVA® by Henkel Corporation, Bridgewater, N.J. (such as DURO-TAK® 87-2516, -2287, -4287, -4098, -2852, -2196, -2296, -2194, -2516, -2070, -2353, -2154, -2510, -9085, -9088 and 73-9301; and GELVA® 2480, 788, 737, 263, 1430, 1753, 1151, 2450, 2495, 3067, 3071, 3087 and 3235). Other suitable acrylic adhesives include those sold under the trademark EUDRAGIT® by Evonik Industries AG, Essen Germany. For example, hydroxy functional adhesives with a reactive functional OH group can be used, including GELVA® 737, 788, and 1151, and DURO-TAK® 87-2287, -4287, -2510 and -2516.

In some embodiments, the drug-containing polymer matrix comprises a silicone-based polymer. The term “silicone-based” polymer is used interchangeably with the terms silicon polymers, siloxane, polysiloxane, and silicones as used herein and as known in the art. A suitable silicone-based polymer may also be a pressure-sensitive adhesive. Thus, in some embodiments, the silicone-based polymer is an adhesive polymer. In other embodiments, the silicone-based polymer functions as an adhesive by the addition of tackifiers, plasticizers, crosslinking agents, or other additives.

Suitable polysiloxanes include silicone pressure-sensitive adhesives which are based on two major components: (i) a polymer or gum and (ii) a tackifying resin. A polysiloxane adhesive can be prepared by cross-linking a gum, typically a high molecular weight polydiorganosiloxane, with a resin, to produce a three-dimensional silicate structure, via a condensation reaction in an appropriate organic, volatile solvent, such as ethyl acetate or heptane. The ratio of resin to polymer can be adjusted in order to modify the physical properties of polysiloxane adhesives. Sobieski, et al., “Silicone Pressure Sensitive Adhesives,” Handbook of Pressure-Sensitive Adhesive Technology, 2nd ed., pp. 508-517 (D. Satas, ed.), Van Nostrand Reinhold, New York (1989).

Exemplary silicone-based polymers are adhesives (e.g., capable of sticking to the site of topical application), including pressure-sensitive adhesives. Illustrative examples of silicone-based polymers having reduced silanol concentrations include silicone-based adhesives (and capped polysiloxane adhesives) such as those described in U.S. Pat. No. Re. 35,474 and U.S. Pat. No. 6,337,086, which are incorporated herein by reference in their entireties, and which are commercially available from Dow Corning Corporation (Dow Corning Corporation, Medical Products, Midland, Mich.) as BIO-PSA® 7-4100, -4200 and -4300 product series, and non-sensitizing, pressure-sensitive adhesives produced with compatible organic volatile solvents (such as ethyl acetate or heptane) and available commercially under their BIO-PSA® 7-4400 series, -4500 series, such as -4502, and -4600 series.

As noted above, in some embodiments the drug-containing polymer matrix comprises one or more rubber-based polymers, such as one or more rubber-based pressure-sensitive adhesives, such as any one or more of those discussed above in the context of the face adhesive.

In accordance with any of the embodiments described herein, the polymer matrix and/or face adhesive layer may comprise an antioxidant. The antioxidant may be one known for use in transdermal drug delivery systems (e.g. the transdermal patch), such as butylhydroxytoluene (BHT), butylhydroxyanisole (BHA), tertiary-butylhydroquinone (TBHQ), ascorbic acid, ascorbyl palmitate, alpha-tocopherol and its esters, fumaric acid, malic acid, sodium ascorbate, sodium metabisulfite, and propyl gallate, and mixtures thereof. The antioxidant may comprise from about 0 to about 1%, including from about 0 to about 0.5% by weight of the polymer matrix face adhesive layer

In accordance with any of the embodiments described herein, the polymer matrix and/or face adhesive polymer may comprise one or more other pharmaceutically acceptable excipients, such as a plasticizer, penetration enhancer, filler, and the like. In some embodiments, the polymer matrix comprises from about 0% to about 20% of one or more such excipients.

A “penetration enhancer” is an agent known to accelerate the delivery of the drug through the skin. These agents also have been referred to as accelerants, adjuvants, and sorption promoters, and are collectively referred to herein as “enhancers.” This class of agents includes those with diverse mechanisms of action, including those which have the function of improving percutaneous absorption, for example, by changing the ability of the stratum corneum to retain moisture, softening the skin, improving the skin's permeability, acting as penetration assistants or hair-follicle openers or changing the state of the skin including the boundary layer.

Illustrative penetration enhancers include but are not limited to polyhydric alcohols such as dipropylene glycol, propylene glycol, and polyethylene glycol; oils such as olive oil, squalene, and lanolin; fatty ethers such as cetyl ether and oleyl ether; fatty acid esters such as isopropyl myristate; urea and urea derivatives such as allantoin which affect the ability of keratin to retain moisture; polar solvents such as dimethyidecylphosphoxide, methyloctylsulfoxide, dimethyllaurylamide, dodecylpyrrolidone, isosorbitol, dimethylacetonide, dimethylsulfoxide, decylmethylsulfoxide, and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin and drugs administered. Other agents include oleic and linoleic acids, ascorbic acid, panthenol, butylated hydroxytoluene (BHT), tocopherol, tocopheryl acetate, tocopheryl linoleate, propyl oleate, and isopropyl palmitate.

In accordance with any of the embodiments described herein, the polymer matrix and/or face adhesive may further comprise one or more various thickeners, fillers, and other additives or components known for use in transdermal drug delivery systems (e.g. the transdermal patch) to further modify properties of the matrix or face adhesive, such as polyvinylpyrrolidone (PVP), crosslinked PVP (crospovidone), polyvinylpyrrolidone/vinylacetate copolymers (PVP/VA, copovidone), ethylene-vinyl acetate copolymers, cellulose derivatives, silica, and other components.

In accordance with any of the embodiments described herein, the polymer matrix may comprise a humectant. Humectants suitable for use in transdermal drug delivery systems (e.g. the transdermal patch) are known, and include PVP, crospovidone, copovidone, and combinations of any two or more thereof. The amount of humectant can be selected based on desired properties, such as an amount effective to impart desired physical properties, such as the adhesion properties of the polymer matrix, such as shear. In some embodiments, a humectant is used in an amount up from about 3% to about 10% dry weight of the polymer matrix, including about 3%, about 5% or about 10% dry weight.

The system may be of any shape or size suitable for continuous systemic application. The polymer matrices and face adhesive layers described herein may be prepared by methods known in the art, and formed into systems by methods known in the art. For example, the polymer matrix material can be applied to a backing layer and release liner by methods known in the art. For example, after the polymer matrix is formed, it may be brought into contact with a support layer, such a releaser liner layer or backing layer, in any manner known to those of skill in the art. Such techniques include calender coating, hot melt coating, solution coating, etc. The face adhesive layer also may be formed on a release liner, and then applied to the polymer matrix layer, and then the systems can be formed into sizes and shapes suitable for use

For example, a polymer matrix can be prepared by blending the components of the polymer matrix, applying the matrix material to a support layer such as a backing layer or release liner, and removing any remaining solvents, and a face adhesive layer can be prepared similarly. The order of steps, amount of ingredients, and the amount and time of agitation or mixing can be determined and optimized by the skilled practitioner. An exemplary general method is as follows:

Appropriate amounts of polymer(s), therapeutic agent(s), other component(s), and organic solvent(s) (for example toluene, or ethyl acetate and/or isopropyl alcohol) are combined and thoroughly mixed together in a vessel. The formulation is then transferred to a coating operation where it is coated onto a protective release liner at a controlled specified thickness. The coated product is then passed through an oven in order to drive off all volatile processing solvents. The dried product on the release liner is then joined to the backing material and wound into rolls for storage. A face adhesive solution containing the face adhesive in a suitable solvent may be coated onto a release liner and dried in a convection oven. The dried face adhesive on the release liner may be laminated with the prepared drug-in-adhesive polymer matrix (after removing its release liner) to form a laminate with a face adhesive. Appropriate size and shape “systems” are die-cut from the roll material and then pouched. Other manufacturing methods are known in the art that are suitable for making the systems described herein.

### Therapeutic Agents

The continuous systemic delivery described herein can be used for formulating any therapeutic agent, such as any therapeutic agent known or determined to be therapeutically active upon continuous systemic delivery. In particular, the therapeutic agent is an agonist of D1-like family of dopamine receptors.

As used herein, the term “dopamine receptors” has its general meaning in the art and refers to a class of G protein-coupled receptors that are prominent in the vertebrate central nervous system (CNS). Dopamine receptors are implicated in many neurological processes, including motivation, pleasure, cognition, memory, learning, and fine motor control, as well as modulation of neuroendocrine signaling. Abnormal dopamine receptor signaling and dopaminergic nerve function is implicated in several neuropsychiatric disorders. There are at least five subtypes of dopamine receptors, D1, D2, D3, D4, and D5. The D1 and D5 receptors are members of the D1-like family of dopamine receptors, whereas the D2, D3 and D4 receptors are members of the D2-like family.

As used herein, the term “D1-like family of dopamine receptors” has its general meaning in the art and refers to the D1-like family receptors coupled to the G protein Gsα. Gsα subsequently activates adenylyl cyclase, increasing the intracellular concentration of the second messenger cyclic adenosine monophosphate (cAMP). The D1-like family of dopamine receptors consists of.
D1 is encoded by the Dopamine receptor D1 gene (DRD1).
D5 is encoded by the Dopamine receptor D5 gene (DRD5).

As used herein, the term “dopamine receptor type 1” (DR1), also known as DRD1, has its general meaning in the art and refers to a protein that in humans is encoded by the DRD1 gene. The naturally occurring human DR1 protein has an amino acid sequence as shown in NCBI accession number: NP_000785.

As used herein, the term “dopamine receptor type 1 (DR1) agonist” refers to a natural or synthetic compound that has a biological effect to increase the activity and/or the expression of dopamine receptor type 1.

As used herein, the term “dopamine receptor type 5” (DR5), also known as D1BR or DRD5, has its general meaning in the art and refers to a protein that in humans is encoded by the DRD5 gene. The naturally occurring human DR5 protein has an amino acid sequence as shown in NCBI accession number: NP_000789.1.

As used herein, the term “dopamine receptor type 5 (DR5) agonist” refers to a natural or synthetic compound that has a biological effect to increase the activity and/or the expression of dopamine receptor type 5.

Examples of therapeutic agents include but are not limited to A-86929, dihydrexidine, dinapsoline, dinoxyline, doxanthrine, SKF-81297, SKF-82958, SKF-38393, fenoldopam, 6-Br-APB, stepholidine, rotigotine, A-68930, A-77636, CY-208,243, SKF-89145, SKF-89626, 7,8-Dihydroxy-5-phenyl-octahydrobenzo[h]isoquinoline, cabergoline, pergolide terguride . . . apomorphine, adrogolide SKF-83959 . . . .

In some embodiment, the therapeutic agent is fenoldopam.

As used herein, the term “fenoldopam” has is general meaning in the rat and refers to a rapid-acting vasodilator having the following structure in the art:

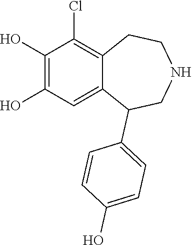 [SMILES: Oc1ccc(C2CNCCc3c2cc(O)c(O)c3Cl)cc1]

The term fenoldopam intends not only the basic form of fenoldopam but also pharmaceutically acceptable salt forms of fenoldopam, the R or S enantiomers of fenoldopam, either individually or as a racemic mixture, and to mixtures thereof.

As used herein, the term “fenoldopam therapy” intends all medical conditions for which fenoldopam is or will be indicated, including, without limitation, for the treatment of hypertension, congestive heart failure, and acute and chronic renal failure.

In some embodiment, the therapeutic agent is rotigotine.

As used herein, the term “rotigotine” has is general meaning in the rat is having the following structure in the art:

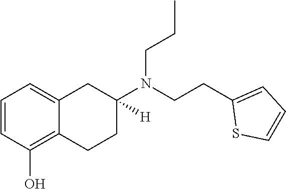 [SMILES: CCCN(CCc1cccs1)[C@H]1CCc2c(O)cccc2C1]

Also provided are methods of effecting continuous systemic delivery of a therapeutic agent (i.e. D1-like family of dopamine receptors agonist), by applying a system as described herein to the skin or mucosa of a subject in need thereof.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In some embodiments, the continuous systemic delivery is formulated to achieve sustained delivery of the therapeutic agent (e.g. the D1-like family of dopamine receptors agonist) over a period of at least about 1 day, at least about 2 days, at least about 3 days, at least about 4 days, at least about 5 days, at least about 6 days, or at least about 7 days, such as for 1, 2, 3, 4, 5, 6 or 7 days or longer if desired. In some embodiments, the method is effective to achieve therapeutic levels of the therapeutic agent during the application period. In some embodiments, the method is effective to achieve a flux decline of less than 30% over the application period.

By a “therapeutically effective amount” is meant a sufficient amount of D1-like family of dopamine receptors agonist for use in a method for the treatment of ADPKD at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of D1-like family of dopamine receptors agonist may be varied over a wide range from 0.01 to 5,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the D1-like family of dopamine receptors agonist for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day. The person responsible for administration will, in any event, determine the appropriate dose for the individual subject.

In some embodiment, the administration by transdermal patch is at a daily dosage of D1-like family of dopamine receptors agonist which may be varied over a wide range from 1 à 20 mg/cm² per adult per day.

In particular, ADPKD patients receive the D1-like family of dopamine receptors agonist using the continuous systemic delivery (e.g transdermal patch or pump) at the dose of 1, 2, 3, 4 or 5 mg per day.

The D1-like family of dopamine receptors agonist as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The invention will be further illustrated by the following figures and examples. However, these examples and figures should not be interpreted in any way as limiting the scope of the present invention.

### FIGURES

FIG. 1: In a prospective parallel study, patients with autosomal dominant polycystic kidney disease (ADPKD) were randomized to receive the dopaminergic receptor agonist rotigotine at the dose of 4 mg per day (n=9) or were not treated (0 mg; n=10). Exploration visits were performed at baseline before (V0), 1 week (V1) and 8 weeks (V2) after randomization. Endothelium-dependent flow-mediated dilatation of the radial artery, assessed by echotracking coupled to Doppler (A), and nitric oxide (NO) release, estimated by the measurement of the NO metabolite nitrite by a chemiluminescent-based assay (B), in response to hand skin heating, aortic pulse pressure (C) did not change during follow-up in non-treated ADPKD patients. However, flow-mediated dilatation increased in patients treated with 4-mg rotigotine and this was associated with an increase in NO release after 8 weeks of treatment. In addition, there was a decrease in aortic pulse pressure after 8 week of treatment with 4-mg rotigotine.

FIG. 2: To specifically assess the role of endothelial polycystin independently from the non-specific consequences of the kidney disease C57/Bl6J mouse transgenic models with an endothelial cell-specific inducible knockout of Pkd1 (VE-Cadh/Pkd1fl/fll) have been developed. Compared to control mice with maintained endothelial expression of polycystin-1 (Pkd1fl/fl), VE-Cadh/Pkd1fl/fl had a decreased mesenteric artery endothelium-dependent dilatation in response to stepwise increase in flow assessed by arteriography (A) and an increased blood pressure measured by tail-cuff plethysmography (B). The administration of the dopaminergic agonist fenoldopam during 14 days using osmotic minipumps (20 mmol/g/day) allowed to improve endothelium-dependent dilatation and to normalize blood pressure in VE-Cadh/Pkd1fl/fl. *P<0.05 vs. VE-Cadh/Pkd1fl/fl.

FIG. 3: Representative scanning electron micrographs of the endothelial surface of the length of the endothelial protrusions, including microvilli and primary cilia obtained in Pkd1^{lox/lox}, iCdh5-cre % ERT2-Pkd1^{del/del} mice receiving or not fenoldopam (4 μg/kg/h) during 4 weeks. Group effect was determined by one-way ANOVA followed by Bonferroni's post hoc test. Three animals per group were used (Pkd1^{lox/lox}: n=265 values; iCdh5-cre1ERT2-Pkd^{del/del}: n=239; iCdh5-cre/ERT2-Pkd1^{del/del}+fenoldopam: n=383). ***P<0.001 vs. Pkdh1^{lox/lox}. ^{†††}P<0.001 vs. Pkd1^{del/del}.

### MATERIAL AND METHODS

### Clinical Study

### Population

ADPKD patients were carefully selected among patients followed in the Departments of Nephrology in Rouen University Hospital, Dieppe Hospital and Le Havre Hospital. The diagnosis was made according to Pei criteria²¹. In addition, molecular testing of ADPKD was performed by direct sequencing, followed by quantitative fluorescent multiplex PCR or array-comparative genomic hybridization²². Only ADPKD patients aged between 18 and 60 years, with a controlled HTN [supine systolic/diastolic blood pressure (BP)<140/90 mmHg] and a glomerular filtration rate (GFR), determined by CKD-EPI formula, >45 ml/min/1.73 m² were included. All patients received the standard-of-care support defined by KDIGO guidelines at inclusion, with BP target <140/90 mmHg (or 130/80 mmHg if presence of diabetes, proteinuria>0.5 g/24 h, heart failure or left ventricular hypertrophy) and use of renin angiotensin system blockers in first intention, body mass index (BMI) target between 20 and 25 kg/m², NaCl intake target <5 g/day, and advice for a regular physical activity.²³

This study was approved by the local Ethics Committee (Committee for the Protection of Persons Nord-Ouest I), and all participants gave written informed consent. The study was conducted in accordance with the principles of good clinical practice and the declaration of Helsinki and was registered at https://eudract.gov under the unique identifier 2016-003393-40.

### Study Design

This was a prospective parallel randomized trial with blind evaluation of primary and secondary endpoints. Ten to thirty days after the visit of inclusion (V1), patients participated to a first exploration visit (V2) in the Department of Pharmacology of Rouen University Hospital and were randomized in groups: a control group of patients on standard care who did not receive rotigotine and a group of patients on standard care and receiving rotigotine at the dose of 4 mg/24 h. According to treatment escalation guidelines, patients randomized in the 4 mg/24 h group received rotigotine at 2 mg/24 h during 7 days and then 4 mg/24 h. A second exploration visit (V3) including safety assessment was performed 7 days after randomization. A phone call was performed for patients receiving the dose of 4 mg/24 h since 7 days to verify tolerance to dose escalation. A third exploration visit (V4) with safety assessment was performed fifty to sixty days after the first one. Then, patients in the 4 mg/24 h group received rotigotine at the dose of 2 mg/24 h during 7 days before stopping treatment. An end of the study visit (V5) was performed in all patients 10 to 20 days after the last exploration visit in the Department of Nephrology of Rouen University Hospital (Data no shown) in particular to verify the tolerance to treatment withdrawal.

### Exploration Visits

On the day of exploration, measurements were performed at least one hour after a fat-free meal while subjects were in a supine position, in a quiet air-conditioned room, maintained at a constant temperature (22 to 24° C.). Subjects were asked to refrain from smoking from the previous evening. A physical examination was performed; brachial artery BP and heart rate were measured on the right arm three times using a brachial cuff oscillometric device (Omron 705IT). Radial artery waveforms of the same arm were recorded non-invasively by applanation tonometry and analyzed by using a dedicated software (Sphygmocor, Atcor Medical), allowing to calculate an averaged radial artery waveform and derive a corresponding central aortic pressure waveform using a validated generalized transfer function.²⁴ Aortic pressure waveforms were subjected to further analysis by the Sphygmocor software to identify the time to the shoulder of the first and second pressure wave components during systole. The pressure at the shoulder of the first component was identified as P1 height (forward pressure wave), and the pressure difference between this point and the maximal pressure during systole (augmentation: ΔP) was identified as the reflected pressure wave. Augmentation index (AIx), an index of cardiovascular coupling, was defined as the ratio of augmentation to central pulse pressure: AIx=(ΔP/PP)×100, where PP is pulse pressure. In addition, carotid-femoral pulse wave velocity, indicator of aortic stiffness, was assessed (Sphygmocor).

Thereafter, the distal skin heating procedure was performed by introducing the right hand in a water-filled thermocontrolled tank.¹³ The device was then filled with water and the temperature was fixed to 34° C. for 20 min to establish baseline conditions. Then, hand skin heating was performed by gradually increasing the water temperature from 34° C. to 44° C., with each level of temperature maintained for 7 min.²⁵ Radial artery diameter and blood flow were measured during the last minute of each step by high-resolution echotracking coupled to a Doppler system (Artlab System, Esaote). The radial artery endothelium-independent dilatation was assessed using 0.3 mg of sublingual glyceryl trinitrate.

Blood samples were drawn at 34° C. for standard biochemistry analyses and the determination of total blood viscosity using a cone-plate viscometer (Ex100 CTB, Brookfield). From the individual values of radial artery diameter (d), flow (Q), and total blood viscosity (μ), the mean arterial wall shear stress was calculated based on a Poiseuille model, that is τ=¼[(4μQ)/(πr³), (r=d/2)].²⁵ In addition, additional local blood sampling was performed at 34 and 44° C. for the quantification of the endothelial factors involved in FMD as previously described.¹³ Plasma levels of nitrite that represent NO bioavailability were determined using a tri-iodide/ozone based chemiluminescence assay. In addition, plasma levels of epoxyeicosatrienoic acids (EETs) and their metabolites dihydroxyecosatrienoic acids (DHETs) were assessed by liquid chromatography coupled to tandem mass spectrometry. EETs+DHETs was used as an index of EETs production.

In addition, at the first and last exploration visits, urinary samples were collected allowing to quantify, using commercially available assays, the levels of cAMP (Cat. No: 581001, Cayman), AQP-2 (Cat. No: MBS2019939, MyBioSource) and copeptin, which is secreted in an equimolar amount to vasopressin, as well as the levels of monocyte chemoattractant protein-1 (MCP-1; Cat. No: DCP00, R&D Systems) that was identified as a prognostic biomarker of renal evolution²⁶. Levels were adjusted to urine creatinine values.

### Statistics

### Sample Size Calculation

The primary endpoint of the study was the change over 2 months in NO release, assessed using plasma nitrite, during hand skin heating. The number of subjects was determined by a nonparametric Kruskall-Wallis test comparing this change between groups, assuming a similar change with a similar variance than previously observed with dopamine infusion (17.3±8.8 nmol/L).¹³ A target sample size of 8 patients in each group was thus needed to demonstrate a significant difference between the control group and at least one treated group with 90% power and a 5% significance level. A maximal number of ADPKD patients to include was fixed to 10 per group based on a trial exit rate not greater than 20%, which is the average rate of trial exits due to adverse events related to 1-year treatment with low-dose rotigotine in patients with restless legs syndrome.^{27,28}

### Analysis

Analyses were performed using the R software (version 4.0.5 et version 1.1.463 of RStudio). Continuous variables are presented as mean±SD in the text, unless differently indicated. Qualitative variables are presented as absolute and relative frequencies. Baseline demographic and clinical data were analyzed using a non-parametric Kruskal-Wallis test, followed by a Dunn test in case of significance, and Fisher's exact tests as appropriate. To answer to the main and secondary objectives, the difference of the variations of the parameters between the first and last exploration visits were calculated for each subject and then compared by a non-parametric Kruskal Wallis test, followed by a Dunn test in case of significance. A Bonferroni correction for multiple comparisons was also used. To characterize and compare the evolution of the parameters over the three exploration visits between groups, a repeated measures analysis was performed. A linear mixed model was estimated with an adapted variance-covariance matrix structure that minimized the AIC and BIC criteria. If the assumptions about the distributions were not met, a nonlinear mixed model could be used by transforming the parameter values in the log scale. On each graph, we presented mean±SEM for the parameter of interest at each exploration visit within each treatment group and p-value of the test for interaction term between time and treatment groups. And if the latter was significant, pairwise comparisons were realized between different exploration visit within each treatment group, taking into account multiple comparisons by a Bonferroni correction for p-values. A value of p<0.05 was considered statistically significant.

### Experimental Studies

### Animals

Experiments were performed in male mice in accordance with the standards and ethical rules, and with approval by the national animal ethics committee (CENOMEXA C2EA-54). Endothelial-specific knock-out of Pkd1 was generated using a tamoxifen inducible cre-loxP recombination system, on a C57BL/6J genetic background as previously described²⁹. Homozygous iCdh5-cre ERT2-Pkd1^{del/del} and control Pkd1^{lox/lox} male mice, all identically exposed to tamoxifen, were used.

### Acute Effect of Fenoldopam

The mesenteric artery relaxation to the peripheral DR agonist fenoldopam (Sigma SM20198) was studied using a small vessel myograph²⁹, before and after addition of the specific antagonist of the DR5 receptor LE-PM436²⁹ in iCdh5-cre ERT2-Pkd1^{del/del} and Pkd1^{lox/lox} mice. In addition, the impact of chronic kidney disease induced by subtotal nephrectomy²⁹ on fenoldopam-induced relaxation was studied in iCdh5-cre ERT2-Pkd1^{del/del} mice. Briefly, after sacrifice, the mesentery was removed and placed in cold oxygenated Krebs buffer. A 1.5-2.0 mm segment of first order of mesenteric resistance artery segment was mounted on a myograph (DMT, Aarhus). After normalization, relaxation to fenoldopam (10⁻⁹ to 10⁻⁵ M) was evaluated in segments precontracted with phenylephrine (Phe: 10⁻⁵ M). Furthermore, the impact of 30-min pretreatment with fenoldopam (10⁻⁶ M) on mesenteric artery FMD was studied in iCdh5-cre ERT2-Pkd1^{del/del}. For this, a 2-3 mm segment of third mesenteric resistance artery segment was mounted on an arteriograph (DMT).^{29,30} The dilatory response to stepwise increases in intraluminal flow (from 3 to 100 μL/min) was assessed in vessels precontracted with Phe (10⁻⁵ M).

### Chronic Effect of Fenoldopam

The impact of 4-week fenoldopam continuous infusion using subcutaneous osmotic pumps (Alzet; 4 μg/kg/h) was studied in iCdh5-cre ERT2-Pkd1^{del/del} mice. A group of Pkd1^{lox/lox} mice was used as control. Before sacrifice, non-invasive measurements of systolic BP were performed by tail-cuff plethysmography (CODA, Kent Scientific Corporation) in conscious and trained mice, and consisted in 2 series of 10 cycles of measurements. After animal sacrifice, mesenteric artery FMD was evaluated in segments precontracted with Phe as described above. In addition, scanning electron microscopy was performed on mouse aortic arch to assess the ultrastructure of the endothelium. Briefly, mice were perfused with a solution of paraformaldehyde 4%, glutaraldehyde 0.5% and HEPES buffer (Sigma) 0.2 mM pH 7.4. Segments of the aortic arch were dissected and fixed overnight in glutaraldehyde 2.5%+HEPES 0.2 mM pH 7.4+lanthanum 2% (Sigma). The samples were thereafter fixed 1 h in OSO4 1% (EMS) at 4° C. Dehydration was performed by graded series of ethanol. Complete dehydration was achieved by immersion in HMDS (Sigma) and air-dried. Samples were fixed on stub with carbon glue and were covered with 10 nm platin (PECS-Gatan-Ametek). Observations were performed on a SEM-JEOL 7900 (JEOL), with 10 kV acceleration, using an Everhart-Thornley electron detector.

### EXAMPLE

### Example 1

### Introduction

Preliminary results obtained using acute local infusion of dopamine (Lorthioir et al, Kidney International 2014) suggested that stimulating peripheral dopaminergic receptors may prevent the alteration in vascular function of patients with autosomal dominant polycystic kidney disease (ADPKD). The aim of this clinical trial was to assess the interest of the chronic stimulation of dopaminergic receptor stimulation at the vascular level

### Methods

A prospective parallel study was performed in patients with autosomal dominant polycystic kidney disease (ADPKD) in the Departments of Clinical Pharmacology and Nephrology of Rouen University Hospital, France. After inclusion, ADPKD patients were randomized to receive the dopaminergic receptor agonist rotigotine continuously administered using transdermal patches at the dose 4 mg per day (n=10) or were not treated (n=9). Exploration visits were performed at baseline before (V0), 1 week (V1) and 8 weeks (V2) after randomization to assess endothelium-dependent flow-mediated dilatation of the radial artery, using echotracking coupled to Doppler, and nitric oxide (NO) release, estimated by the measurement of the NO metabolite nitrite using a chemiluminescent-based assay. In addition, aortic pulse pressure was measured using applanation tonometry.

### Results

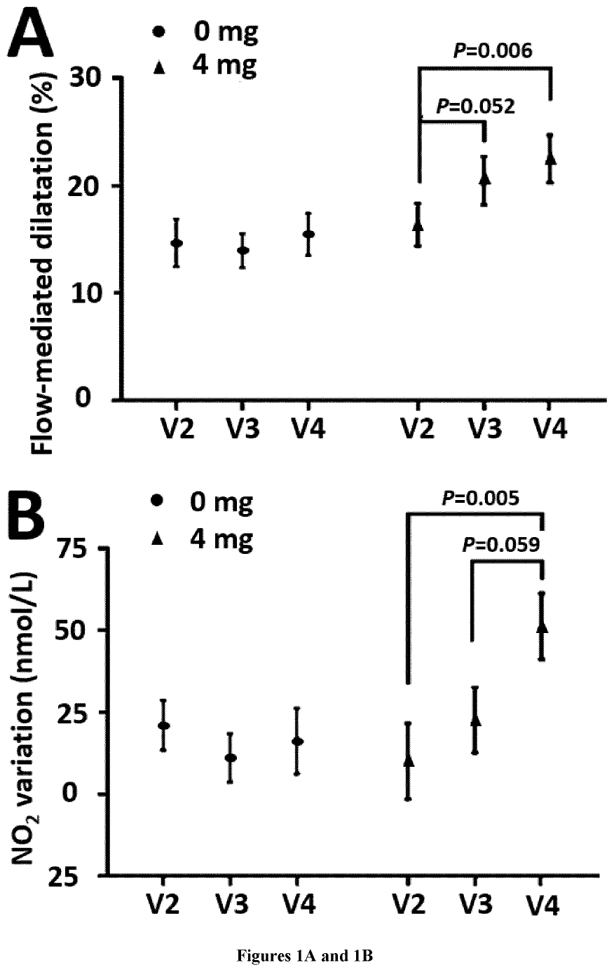

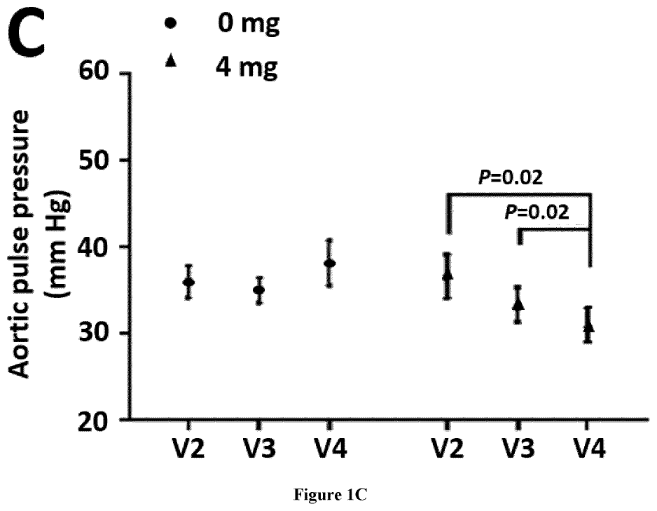

FIGS. 1A-1C: Endothelium-dependent flow-mediated dilatation, NO release and blood pressure did not change during follow-up in non-treated ADPKD patients. However, flow-mediated dilatation increased in patients treated with 4-mg rotigotine and this was associated with an increase in NO release after 8 weeks of treatment. In addition, there was a decrease in aortic pulse pressure after 8 week of treatment with 4-mg rotigotine.

### Conclusion

These results demonstrate that the chronic stimulation of dopaminergic receptors in ADPKD patients with rotigotine improves vascular endothelial function and central hemodynamics, representing thus a new therapeutic option for the clinical management of hypertension and cardiovascular complications in this population.

### Example 2

### Introduction

Preliminary results obtained using acute local infusion of dopamine (Lorthioir et al, Kidney International 2014) suggested that stimulating peripheral dopaminergic receptors may prevent the alteration in vascular function of patients with autosomal dominant polycystic kidney disease (ADPKD). The aim of this experimental study was to assess the interest of the chronic stimulation of dopaminergic receptor stimulation independently from non-specific consequences of this approach on kidney disease.

### Methods

C57/Bl6J mouse transgenic models with an endothelial cell-specific inducible knockout of Pkd1 (VE-Cadh/Pkd1fl/fll) have been developed and these mice were treated with the dopaminergic agonist fenoldopam during 14 days using osmotic minipumps (20 mmol/g/day). Mesenteric artery endothelium-dependent dilatation in response to stepwise increase in flow was assessed by arteriography and blood pressure was measured by tail-cuff plethysmography.

### Results

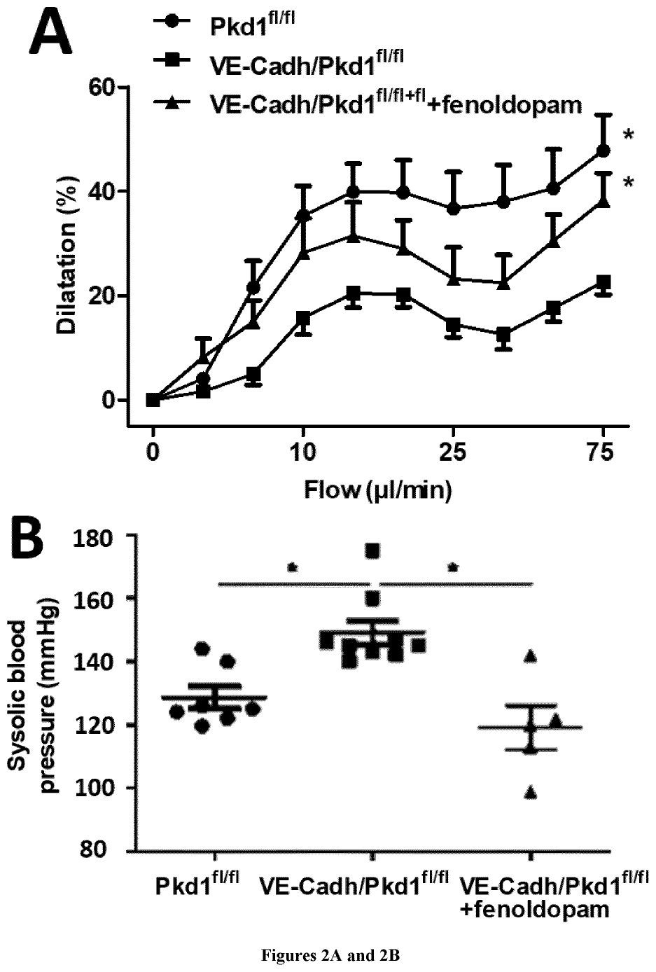

FIGS. 2A-2B: Compared to control mice with maintained endothelial expression of polycystin-1, mice with endothelial deletion of polycystin-1 had a decreased endothelium-dependent dilatation and an increased blood pressure. The administration of fenoldopam allowed to improve endothelium-dependent dilatation and to normalize blood pressure in mice with endothelial deletion of polycystin-1.

### Conclusion

These results demonstrate that the chronic stimulation of dopaminergic receptors with fenoldopam improves vascular function and decreases blood pressure in mice with a specific endothelial deletion of polycystin-1, representing thus a new therapeutic option for the clinical management of hypertension and cardiovascular complications of ADPKD patients.

### Example 3

### Chronic Effect of Fenoldopam

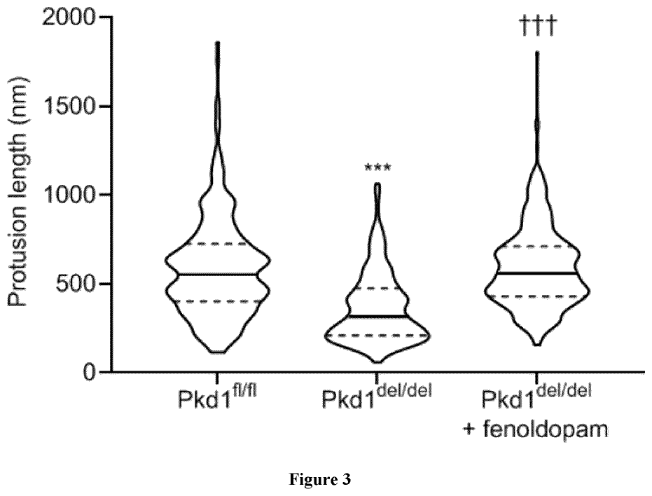

Scanning electron microscopy analysis revealed the presence of protrusions at the endothelial cell surface of the aortic arch of control mice that were mainly located in specific regions thought to be dependent of a high level shear stress (Data not shown). It was not possible to formally discriminate microvilli from primary cilia but, as previously described,¹⁴ microvilli seemed to predominate. Endothelial polycystin-1-deficient mice exhibited protrusions that were characterized by a stockier and more compact aspect compared to the control mice. In endothelial polycystin-1-deficient mice treated with fenoldopam, protrusions showed a linear aspect alike protrusions of control mice. Thus, fenoldopam corrected the decrease in the length of endothelial protrusions associated with endothelial polycystin-1-deficiency (FIG. 3).

### REFERENCES

Throughout this application, various references describe the state of the art to which this invention pertains. The disclosures of these references are hereby incorporated by reference into the present disclosure.
1. Massella L et al, CJASN 2018; 13(6):874-883;
2. Chebib F T et al, Kidney Int Rep 2017; 2(5):913-923;
3. Reinecke N L et al, AJKD 2014; 64(2):239-246;
4. Weyde W et al, Clin Nephrol 2004; 61(5):344-6;
5. Su Q et al, Science 2018; vol. 361(6406);
6. Harris P C et al, J Clin Invest 2015; 124(6):2315-2324;
7. Sharif-Naeini R et al, Cell 2009; 139(3):587-596;
8. Nauli S M et al, Circulation 2008; 117(9):1161-71;
9. AbdouAlaiwi W A et al, Circ Res 2009; 104(7):860-9;
10. Lorthioir A et al, Kidney Int 2015; 87(2):465-72;
11. Yamamoto K et al, J Vis Exp 2013; 11(77):e50449; 1
2. Schrier R W et al, JASN 2014; 25(11):2399-2418;
13. Gildea J J et al, Kidney Int 2014; 86(1):118-126
14. Van der Heiden, K, et al, Dev. Dyn. 2006; 235:19-28.

1. A method of treating autosomal dominant polycystic kidney disease (ADPKD) in a subject in need thereof comprising a step of administering said subject with a therapeutically effective amount of a D1-like family of dopamine receptors agonist through a continuous systemic delivery.

2. The method according to claim 1 wherein D1-like family of dopamine receptors agonist is fenoldopam.

3. The method according to claim 1 wherein D1-like family of dopamine receptors agonist is rotigotine.

4. The method according to claim 1 wherein the continuous systemic delivery is administered with a pump.

5. The method according to claim 5 wherein the pump is an osmotic pump.

6. The method according to claim 1 wherein the continuous systemic delivery is administered with a transdermal patch.

7. The method according to claim 6 wherein the transdermal patch comprises a backing layer and a release liner.

8. The method according to claim 6 wherein the transdermal patch comprises a face adhesive.

9. The method according to claim 1 wherein the continuous systemic delivery achieve sustained delivery over a period of at least about 1 day, at least about 2 days, at least about 3 days, at least about 4 days, at least about 5 days, at least about 6 days, or at least about 7 days.
